(19) United States
(12) Patent Application Publication
(10) Pub. No.: US 2009/0124902 A1
(43) Pub. Date: May 14, 2009

(54) Method for in vivo tissue classification

(76) Inventor: Vera Herrmann, Luebeck (DE)

Correspondence Address: K.F. ROSS P.C., 5683 RIVERDALE AVENUE, SUITE 203 BOX 900, BRONX, NY, 10471-0900, US

(21) Appl. No.: 11/988,675

(22) PCT Filed: Jul. 7, 2006
(86) PCT No.: PCT/DE2006/001174

§ 371 Date: Jul. 24, 2008

(30) Foreign Application Priority Data
Jul. 19, 2005 (DE) 10 2005 034 219.1

(51) Int. Cl. A61B 8/00; A61B 5/00

(52) U.S. Cl. 600/437; 600/476

(57) ABSTRACT

The invention relates to a method for the classification of tissue from the lumbar region, using an ultrasonic transducer array comprising a control device, at least one light source with a small spectral width in a wavelength range above 500 nm, at least one light detector, and a process computer for processing the measuring values of the light detector. According to the invention, the light detector detects only backscattered light from the tissue, the ultrasonic transducer array injects focussed ultrasounds into the tissue during the illumination thereof, and the process computer isolates the contribution of the ultrasound focus of the scattered light from the total light intensity measured by the light detector and calculates optical parameters therefrom for the tissue in the ultrasound focus. The process computer derives a characteristic variable from the calculated parameters, which is optimised in terms of a pre-determined optimality criterion, by controlling the control device in such a way that the position of the ultrasound focus is modified in the tissue according to said process computer, and the process computer compares the optical parameters in the optimum position of the ultrasound focus with a stored data table, thus being able to classify the tissue.

The invention relates to a method of in vivo tissue classification in which ultrasound and infrared light are radiated into living tissue, particularly into the human or the animal body, and the re-emerging light is used to determine local optical parameters, particularly the absorption and/or backscattering ability of the tissue, thus allowing a classification of the tissue.

Ultrasonic examinations for the purpose of locating abnormal tissue in a living organism have been part of the prior art for a long time. A conventional application is the area of mammography, which is to say the detection of breast cancer in women. Malignant tissue, particularly cancerous tissue, is characterized among other things by different mechanical properties than the surrounding healthy tissue, so that during the ultrasonic examination impedance contrasts at the interfaces result in reflection of the sound waves. This characteristic is used to locate abnormal tissue. An ultrasonic examination alone, however, does not allow any conclusion yet as to whether the abnormal tissue discovered is a malignant tumor or not. As a result, a common procedure is the removal of a sample of the tumor (biopsy) for definitive determination in the laboratory.

Based on the samples taken, it is not only possible to precisely classify the tissue, but also to accurately measure the optical properties thereof. In particular it has been found that cancerous cells absorb certain wavelengths of the near-infrared (NIR) and mid-infrared (MIR) spectra considerably more strongly than healthy cells.

The state of the art that should be mentioned is 103 11 408 [U.S. Pat. No. 7,251,518] by the inventor.

Due to the water band minima, the human body is largely transparent in the wavelength range between approximately 600 and 1000 nm (“biological window”), which is to say that light can penetrate deep into the tissue, can pass through it, or also return to the irradiated surface. There are further “transparent windows” in the MIR spectrum that are characterized by low water absorption compared to other tissue components, for example between 5000 and 7500 nm and even between 10 and 25 micrometers.

Within such “transparent windows,” it is possible to specify for each individual tissue component a light-wave length that is easily absorbed or scattered by this tissue portion. From tumor tissue taken from ex vivo examinations it is already known that some wavelengths are particularly characteristic for cancer cells, in part because these comprise certain substances that do not occur in healthy tissue.

WO 1994/028795 [U.S. Pat. No. 6,002,958] proposes a method of performing an in vivo tissue classification by the combined radiation of a focused ultrasound beam and NIR light. The transmitted and/or backscattered radiation in the wavelength range of 600 to 1500 nm leaving the tissue serves as a measurement signal, wherein the radiation changes as the ultrasound focus is displaced through the tissue. The displacement of the focus is possible, for example, by the suitable control of a transducer array, as that described for example in U.S. Pat. No. 5,322,068.

WO 1994/028795 in detail teaches that the focus should be displaced continuously in three dimensions through the tissue to be examined in order to pass through both normal and abnormal tissue so that the abnormal tissue can be classified based on the “contrast” with the normal tissue; the focused ultrasonic beam should be applied in an amplitude-modulated manner in order to assess the tissue with respect to the mechanical parameters (for example relaxation time) based on the influence of the varying amplitude on the light signal the focus position should be held stationary in a point exhibiting significant influence of the ultrasound amplitude on the optical signal so as to vary the spectral composition of the irradiated NIR light; a conclusion can be drawn of the tissue pathology from the dependency of the optical measurement signal on the spectral composition.

All of the above measures are certainly reasonable and possibly necessary to arrive at a comprehensive biophysical analysis of the complex cell tissue. It is known, for example, that living cells change their optical properties under pressure and as a function of the temperature. As a result, detailed variation analyses are certainly the tool of choice in order to appropriately take all significant influencing factors affecting the optical measurement signal into consideration.

In medical practice, the question of interest however is initially much simpler: Should suspicious tissue that is detected during the ultrasound examination be removed and examined in the laboratory, or is this perhaps avoidable?

In general, a biopsy is quite unpleasant or even painful for the patient, however it is associated with little effort for the treating physician. The comprehensive measurement according to WO 1994/028795 for the purpose of a medical diagnosis is rather disadvantageous because
the continuous displacement of the ultrasonic beam focus alone (volume<1 mm³) through a three-dimensional measuring region that is at least 1000 times larger can be done only slowly and is therefore time-consuming;
the observation of cell-mechanical parameters for locating malignant areas appears rather complex compared to the conventional ultrasonic reflection measuring method, even if it allows perhaps for more precise mapping, which may not necessarily be of interest to the physician (at least not for early detection of cancer);
the variation of the spectral composition of the measuring light requires variable light sources and/or spectral analyzers, which per se are already expensive components, so that the proposed apparatus is associated with considerable procurement expenses.

In addition to these disadvantages, the apparatus according to WO 1994/028795 is primarily designed for the detection of transmitted light, although a one-sided measuring device measuring only backscattered light is explicitly mentioned. Backscattered light, however, is generally subjected to multiple scattering steps, which is to say it travels a relatively unpredictable path from the light source to the detector disposed adjacent thereto. As a result, it is also uncertain whether the returning light perhaps passed through the ultrasound focus. In other words, pure backscattering is subject to the problem of source localization for the contributions to the optical measuring signal not solved by WO 1994/028795.

Patent DE 103 11 408 [U.S. Pat. No. 7,251,518] mentioned above, however, describes a possibility for non-invasively determining the concentration of blood components from the backscattering of special IR wavelengths, where an ultrasonic beam is focused on the inside of a blood vessel to mark the backscatter region. The evaluation method is designed to differentiate the light returned from the focus from the remaining backscattered light and to determine optical characteristics only for the focus region. The apparatus according to DE 103 11 408 uses a plurality of IR laser diodes whose wavelengths are adjusted right from the start to the task at hand, particularly to the measurement of blood oxygen. The apparatus is not suited without modification for general tissue examinations because it relies, among other things, on finding a suitable focus position based on the Doppler principle, wherein it assumes the presence of a sufficiently high volume of blow flowing with a focus.

It is therefore the object of the invention to further develop the state of the art such that a simplified apparatus for non-invasive in vivo tissue classification is obtained.

The object is solved by a apparatus having the characteristics of claim 1. The dependent claims describe advantageous embodiments.

The inventive apparatus comprises an ultrasonic device that is configured as a transducer array having an electronic controller and that can emit and receive ultrasound. Depending on activation, the source can optionally emit ultrasound having substantially flat, concave, or convex wave fronts, which is to say it can send radiation into the tissue to be examined particularly in a fanned or focused manner. The focus position can be selected and can be varied by the controller during the measurement based on external specifications. Furthermore, the controller can use the propagation time measurement of sound waves reflected in the tissue to draw a conclusion of a spatial target area comprising a tissue abnormality.

The inventive apparatus furthermore comprises one or preferably more light sources having close spectral distribution, laser diodes being particularly preferred. The number of light sources and the selection of the respective main emission wavelength shall remain variable, so that a modular design is recommended. Alternatively, and certainly also as a function of the future price development of these light sources, also a larger number (for example 10-20 different wavelengths) of sources may be provided on the apparatus at any given time, in which case the sources of course must be individually switchable.

In principle, all wavelengths from the NIR and MIR spectral ranges are of interest, which is to say in concrete non-ionizing radiation having a wavelength of at least 500 nm. For the selection of wavelengths for the in vivo measurement, of course, not just any arbitrary microwave beams will or can be used, in particular lasers will not be available for every wavelength of interest. The primary focus here shall therefore be aimed at the “biological window” (500-1000 nm), however the invention shall not be interpreted as being limited thereto. It may certainly be expedient to classify certain tissue types based on wavelengths far outside the biological window.

Furthermore, the apparatus according to the invention includes a light detector, particularly advantageously a flat, light-sensitive sensor array (such as a CCD camera) that measures the backscattered light intensity. The light detector is read by an electronic process computer at regular intervals. The process computer additionally uses the parameters of the ultrasonic field supplied by the ultrasonic controller, particularly sound frequency, pulse energy, and repetition rate. With the help of the algorithm already outlined in DE 103 11 408, the portion of the light backscattered in the region of the ultrasound focus is isolated from the total light intensity.

Taking the known depth of the focus under the tissue surface into consideration, scattering loss of the isolated light portion typically found in healthy tissue can be compensated for in the computer. Following compensation, a value is computed, for example for the absorption coefficient and/or for the backscattering capacity of the tissue on the inside of the ultrasound focus, wherein the value can refer to individual or a plurality of wavelengths at the same time.

For tissue classification it is necessary to adjust the focus position to the most meaningful position in any detectable abnormal tissue. This position does not necessarily coincide with the center of the region located by ultrasonic scanning having modified acoustic impedance. In the presence of pathologically modified cells, the abnormality is rather characterized by abnormal cell chemistry and is thus detectable above all based on the optical parameters.

According to the invention, the focus position thus is modified fully automatically based on the respectively measured absorption and/or backscattering of the tissue in the focus. The focus position does not require continuous displacement, but can be changed randomly. The comparison of the absorption and/or dispersion coefficient at a defined focus position with that of one or more prior positions allows a conclusion by algorithm of a successive position that is adjusted during the next measuring process by the ultrasonic controller.

The selection of a sequence of focus positions by algorithm is nothing other than a simple optimization problem. It means the search for the ideal location for a characteristic variable of one or more light-wave lengths within the abnormal tissue previously discovered by ultrasound, the characteristic variable being derived from the measurable absorption and/or scattering. Which characteristic variable is used or which ideal location is desired will depend on the concrete task.

A preferred proposition is to determine the variance of the absorption or dispersion coefficients in the focus from those in healthy tissue as the characteristic variable (a reference that is recorded at the beginning of the measurement process) and to search for the local maximum thereof.

Attention will primarily be directed at absorption, for example, if the patient was previously administered a dye that accumulates primarily in malignant tissue. In such a case, advantageously the irradiated light-wave lengths are those that easily absorb the dye. When using such a selective dye, the recording of a reference for healthy tissue can even be foregone. For other areas, such as the examination of fatty tissue, the analysis of backscattering is more meaningful.

The selection of the characteristic variable to be used is relatively apparent for every problem and the user will be aware that the optimum (here the maximum) can exist in any position in the tissue. In addition, it can be assumed that the function to be maximized is consistent and assuming the differentiability of the function will be justified, so that for example a gradient decline or any other known optimization algorithm can be used to compute the sequence of the focus positions (interpolation points of the function).

The precise algorithm that is used to compute the optimization is not relevant here. More important is the inventive idea that the displacement of the ultrasound focus occurs based on the portion of the backscattered light intensity that was previously associated solely with the tissue of the focus region. The focus is automatically displaced until it arrives at an optimal meaningful position in the tissue.

Once this position has been recorded with the ultrasound focus, it is recommended to individually determine the absorption coefficients (and/or backscatter coefficients) for all available IR wavelengths. The process computer should additionally comprise a data table that is used to compare the measurement results. The table comprises the largest possible number of tissue types, including the respective known optical parameters, as those measured in the laboratory, for example. This will provide the user of the measuring apparatus directly with a tissue classification. However, attention must be paid to the fact that the data tables available according to the current state of the art are based on pathological findings, which is to say that extracted tissue samples were measured, which certainly will differ significantly with respect to the temperature, pressure, pH value, or blood components in the surrounding area of the in vivo situation. This will considerably influence the optical parameters.

Nevertheless, it can be assumed that the cell chemistry remains largely unaffected, so that a reasonable classification within certain tolerance limits is possible. Determining the extent of such tolerances will require future, particularly empirical work. However, it is already apparent now that a deviation of the optical parameters obtained according to the invention from those determined based on the pathological samples is practically unavoidable and that therefore only a probability statement can be made about the classification of the tissue.

Computing this probability in concrete terms and making it available to the user is a particularly preferred embodiment of the invention.

Contrary to 102 11 403, according to which a classification of living tissue is performed, which is based on a combination of infrared analysis and focused ultrasound, the ultrasound focus is positioned as a function of the results of the optical measurement.

For example, a tuple of measured values (A1, A2, R3, A4, . . . ) can be such an optical parameter, where for example A1 denotes the absorption coefficient for wavelength 1 and R3 the backscatter coefficient for wavelength 3. The essential aspect is that the optical parameters for a fixed focus position are first measured. In order to optimize the measurement, the process computer will then propose a better focus position that is controlled by the ultrasonic transducer array. The actual optical measured values of the second focus position are recorded and included in a new assessment of the process computer, and so on.

In this way, iteratively and automatically a maximally meaningful focus position is discovered (without the gradual displacement through the tissue, which would be extremely time-consuming), where the classification is performed.

Tissue classification following optimal positioning of the ultrasound focus according to the methods described in the application is subject to the requirement that the optical signal detected at the light detector allows a direct conclusion of optical tissue parameters on the current focus position.

Particularly for backscattered light, the precise source localization is nontrivial due to the multiple scattering of photons in the living tissue. While the optical measuring signal is used for substance analysis in the patent mentioned, focus positioning relies on the use of the acoustic Doppler effect in the presence of a sufficient amount of blood with high flow. The use in any arbitrary tissue outside of the large blood vessels, however, is not described.

The invention will be explained in more detail hereinafter based on the only figure:

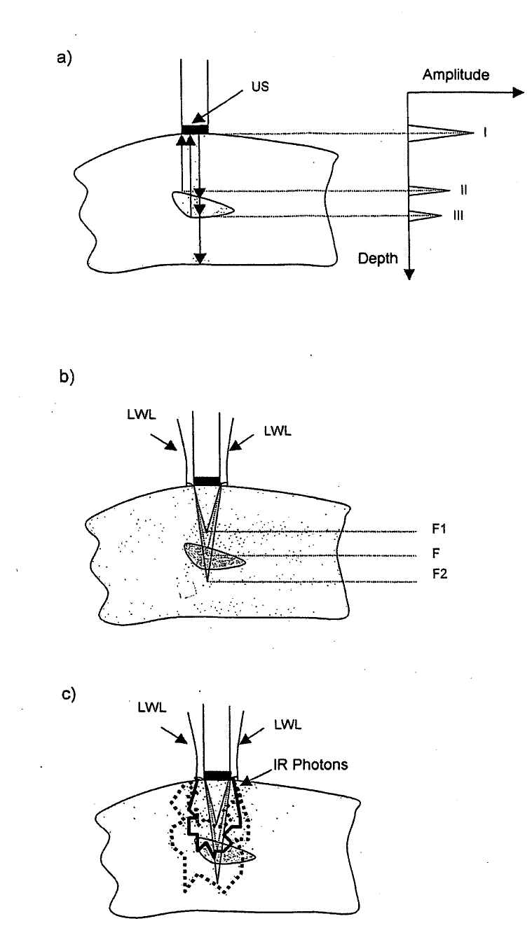

FIG. 1 is a schematic illustration of the procedure implemented in the apparatus for locating the most meaningful focus position for tissue classification.

In the preferred embodiment of the inventive apparatus, an ultrasonic transducer array, a plurality of light sources, and a light-sensitive sensor array are positioned adjacent one another and integrated in a hand-held applicator. The light sources and sensor array are preferably positioned concentrically around the transducer array. The applicator should preferably be fastened to the surface of the tissue to be examined (patient's skin), for example by a vacuum or a medical adhesive.

As is shown in FIG. 1a, the applicator begins the examination by means of tissue scanning in order to locate regions of interest based on impedance contrasts. The transducer array (ultrasonic) first applies fanned ultrasound, and the propagation times of the reflected signals are determined by the controller. These propagation times are converted into coordinates of the tissue that is to be analyzed and may be abnormal. From the coordinates, the control parameters of the individual transducer elements are determined in the known manner, the parameters allowing the generation, and optionally the displacement, of an ultrasound focus in the target area comprising the abnormal tissue. The coordinates of the target area are likewise transmitted to the process computer that is responsible for reading the optical sensor array and computing the optical parameters.

After determining the target area, light having low spectral width, preferably laser light, is irradiated into the tissue, an ultrasound focus being formed at the same time. In FIG. 1b, the light is conducted via optical fibers (LWL) adjacent the ultrasound source, whence it enters the tissue. The light sources therefore do not necessarily have to be integrated into the applicator, but only the means for guiding the light. FIG. 1b furthermore shows that two focus positions in the depths F1 and F2 are set up outside of the target area in order to record the optical parameters of the healthy tissue for reference purposes. Recording a reference at the start of a classification procedure is typically necessary and always recommended, already because different patients differ significantly from one another and even on the same patient time dependence of the measuring results may exist (such as repeated measurements on different days).

The function to be maximized algorithmically in this case is to define the variances of the measured values in the target area from those of the normal tissue. For this purpose, the backscattered light intensities are measured by the sensor array and are divided by the process computer into portions that have passed the ultrasound focus and those that have not, and the optical parameters of the focus region are computed. Based on the coordinates of the current focus position transmitted by the controller, a numeric function is obtained in the process computer, this function being scannable by interpolation points. Since here only the maximum of the function is desired, scanning can be performed erratically using known optimization algorithms. The process computer directly uses the optical measuring data and the algorithms to command the controller to reposition the focus for the next interpolation point.

The iteration of the focus position ends automatically as soon as the focus is located in the tissue with the highest abnormality. It may be advantageous to provide further convergence-forcing criteria in the program, for example in the simplest case stopping the iteration based on a predetermined number of iteration steps.

In the concrete example of FIG. 1, two initial measuring sites are adjusted in the depths F1 and F2. The measured values can be averaged, for example, and may serve as reference values for normal tissue. Likewise, a third measured value can be determined for a focus position in the target area (depth F), the value being compared separately to the two values at F1 and F2. The selection and number of the initial focus positions depends among other things on the iteration algorithm and should therefore not be interpreted as a limiting factor for the invention. For some optimizing algorithm it may be particularly advantageous to select the initial interpolation points randomly.

FIG. 1c shows a schematic illustration of some scatter paths of irradiated IR photons that return to the optical fiber (LWL) where they each pass through a focus. In principle, the photons can re-enter the optical fiber and be directed to a detector. Already for reasons of lowest backscattered intensity, however, it is preferable to place a flat sensor array as a light director directly on the tissue to be examined (not shown) and record the intensity in an integrating manner across all array elements. The sensor array should under any circumstances have a lateral extension that takes into account that the returning light tends to exit with more lateral offset the deeper it is scattered in the tissue. This empirically known correlation can incidentally be used to isolate the light backscattered in the ultrasound focus, because the depth of the focus is always known.

In summary, the inventive apparatus achieves the following two tasks:
It uses ultrasound and backscattered IR light to fully automatically locate a position of the ultrasound focus that has the best possible meaning for tissue classification based on optical parameters by means of an implemented optimization algorithm.
It examines the tissue in the previously ideally positioned ultrasound focus—and only there—with respect to the optical parameters for a plurality of predetermined IR wavelengths and, based on the comparison of these measured values, performs a classification of the tissue under review using tabulated findings from pathological examinations.

Ideally, simply already because of the above-described variances between in vivo tissues and extracted tissue samples, the process computer—in addition to the classification—provides probability information about the accuracy of the analysis in order to support the treating physician in the decision about further steps.

An advantageous embodiment of the invention is to specifically store the measured parameters if the physician decides in favor of sampling tissue and a laboratory examination. The laboratory results can then be entered via an interface, such as a screen-based entry program on the process computer, together with the stored measured data in order to gradually expand the data inventory used for the classification.

1. A method of in vivo tissue classification of living tissue, using an ultrasonic transducer array, a controller for the transducer array, at least one light source having low spectral width in the wavelength range above 500 nm, at least one light detector, and a process computer for processing the measured values of the light detector, the light detector detecting only light backscattered from the tissue, the ultrasonic transducer array during the illumination irradiating focused ultrasound into the tissue, and the process computer isolating the contribution of the light scattered in the ultrasound focus from the total light intensity measured by the light detector and computing therefrom optical parameters for the tissue in the ultrasound focus wherein the process computer uses the computed parameters to derive a characteristic variable that is optimized with respect to a predefined optimization criterion, in that the position of the ultrasound focus in the tissue is modified by the controller as specified by the process computer, and that the process computer compares the optical parameters in the discovered optimal position of the ultrasound focus with a stored data table and thus classifies the tissue.

2. The method according to claim 1, wherein the characteristic variable is the variance of the optical parameters from the reference values in healthy tissue recorded during the measurement.

3. The method according to claim 1 wherein the predefined optimization criterion is the maximization of the characteristic variable.

4. The method according to claim 1 wherein prior to measuring the optical parameters an ultrasound scanning step is performed, during which the controller records the propagation times of reflected sound waves and that based thereon a region of the tissue to be classified is determined in which the ultrasound focus is to be formed.

5. The method according to claim 1 wherein the data table stored in the process computer comprises tissue classifications and the optical parameters thereof from ex vivo measurements.

6. The method according to claim 5 wherein the process computer optionally stores measured optical parameters and comprises a user interface, via which tissue classifications can be associated with the stored parameters, wherein the stored data table is updated.

7. The method according to claim 1 wherein, during the comparison of the optical parameters with the stored data table, the process computer computes and issues a probability for the accuracy of the classification.
